(19) United States
(12) Patent Application Publication
(10) Pub. No.: US 2008/0262060 A1
(43) Pub. Date: Oct. 23, 2008

(54) Crystalline forms of Deferasirox

(76) Inventors: Zoltan G. Toth, Debrecen (HU); Tivadar Tamas, Debrecen (HU); Csilla Nemethne Racz, Tiszavasvari (HU)

Correspondence Address: KENYON & KENYON LLP, ONE BROADWAY, NEW YORK, NY, 10004, US

(21) Appl. No.: 12/011,999

(22) Filed: Jan. 29, 2008

Related U.S. Application Data
(60) Provisional application No. 60/898,368, filed on Jan. 29, 2007.
(60) Provisional application No. 60/919,428, filed on Mar. 21, 2007.
(60) Provisional application No. 60/994,223, filed on Sep. 17, 2007.

(51) Int. Cl. A61K 31/4196; C07D 249/08

(52) U.S. Cl. 514/383; 548/269.4; 548/269.2

(57) ABSTRACT

The present invention provides novel crystalline forms of deferasirox, methods for their production, and methods for conversion of the novel forms to the known crystalline form I.

### CROSS REFERENCE TO RELATED APPLICATIONS

This application claims priority of U.S. provisional application No. 60/898,368 filed Jan. 29, 2007; U.S. provisional application No. 60/919,428 filed Mar. 21, 2007, and U.S. provisional application No. 60/994,223 filed Sep. 17, 2007. The contents of these three applications are incorporated by reference herein for all purposes, and in their entirety.

### FIELD OF THE INVENTION

The present invention relates to crystalline forms of Deferasirox, methods for the preparation thereof, and pharmaceutical compositions thereof.

### BACKGROUND OF THE INVENTION

4-[3,5-bis(2-hydroxyphenyl)-1H-1,2,4-triazol-1-yl]benzoic acid, Deferasirox (referred to as DFX) of the formula

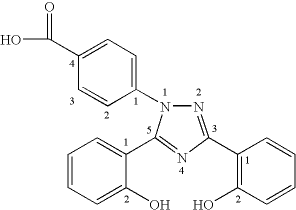

is a tridentate ligand that selectively binds Fe³⁺ ions in a 2:1 ratio. DFX is primarily used for the treatment of chronic iron overload due to blood transfusions (transfusional hemosiderosis).

DFX is marketed under the trade name Exjade® by Novartis Pharmaceuticals Corp.

DFX and process for preparing it are disclosed in WO 97/049395. The process includes reacting 2-(2-hydroxyphenyl)benz[e][1,3]oxazin-4-one and 4-hydrazinobenzoic acid in boiling ethanol, where upon cooling crystals are obtained.

Publication number IPCOM000146862D describes a crystalline form of DFX, designated form I, characterized by X-ray powder diffraction having peaks at about 13.2, 14.1 and 16.6±0.2 degrees 2θ. Form I may be further characterized by X-ray powder diffraction having peaks at about 6.6, 10.0, 10.6, 20.3, 23.1, 25.7 and 26.2±0.2 degrees 2θ, and by an X-ray powder diffraction pattern depicted in FIG. 1.

Crystalline DFX is also disclosed in Complex formation of ICL670 and Related Ligands with Fe^{III} and Fe^{II} (S. Steinhauser, et al. Eur. J. Inorg. Chem., 2004, 4177-4192).

Polymorphism, the occurrence of different crystal forms, is a property of some molecules and molecular complexes. A single molecule, like DFX, may give rise to a variety of crystalline forms having distinct crystal structures and physical properties like melting point, x-ray diffraction pattern, infrared absorption fingerprint, and solid state NMR spectrum. One crystalline form may give rise to thermal behavior different from that of another crystalline form. Thermal behavior can be measured in the laboratory by such techniques as capillary melting point, thermogravimetric analysis (“TGA”), and differential scanning calorimetry (“DSC”), which have been used to characterize crystal forms.

The present invention relates to the solid state physical properties of DFX. These properties can be influenced by controlling the conditions under which DFX is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance that can be identified unequivocally by X-ray diffractometry. The polymorphic form may give rise to thermal behavior different from that of the amorphous material or another polymorphic form. Thermal behavior is measured in the laboratory by such techniques as capillary melting point, thermogravimetric analysis (TGA) and differential scanning calorimetry (DSC) and can be used to distinguish some polymorphic forms from others. A particular polymorphic form may also give rise to distinct spectroscopic properties that may be detectable by solid state ¹³C NMR spectrometry and infrared spectroscopy.

The difference in the physical properties of different crystalline forms results from the orientation and intermolecular interactions of adjacent molecules or complexes in the bulk solid. Accordingly, polymorphs are distinct crystalline forms sharing the same molecular formula yet having distinct physical properties as compared to other crystalline forms of the same compound or complex. These distinctive physical properties may, alone or in combination, confer advantages to a particular polymorph in pharmaceutical applications.

One of the most important physical properties of pharmaceutical compounds is their solubility in aqueous solution, particularly their solubility in the gastric juices of a patient. For example, where absorption through the gastrointestinal tract is slow, it is often desirable for a drug to dissolve slowly so that it does not accumulate in a deleterious environment. This is particularly true when the drug is unstable to conditions in the patient's stomach or intestine. Different crystalline forms or polymorphs of the same pharmaceutical compound can (and reportedly do) have different aqueous solubility.

The discovery of new polymorphic forms of a pharmaceutically useful compound provides a new opportunity to improve the performance characteristics of a pharmaceutical formulation. It also enlarges the repertoire of materials that a formulation scientist has available for designing a pharmaceutical dosage form of a drug such as a targeted release profile or other desired characteristic. Because of limited options, there is a need in the art for novel polymorphic forms of DFX, such as those presented below.

### SUMMARY OF THE INVENTION

One embodiment of the invention encompasses crystalline DFX characterized by data selected from the group consisting of: a PXRD pattern having peaks at about 5.3, 10.6, and 13.9±0.2 degrees 2θ, a PXRD pattern depicted in FIG. 2, and combination thereof.

Another embodiment of the invention encompasses a process for preparing crystalline DFX characterized by data selected from the group consisting of: a PXRD pattern having peaks at about 5.3, 10.6, and 13.9±0.2 degrees 2θ, a PXRD pattern depicted in FIG. 2, and combination thereof comprising providing a solution of DFX in water having a basic pH, and reducing the pH to obtain an acidic pH, thus providing the said crystalline DFX.

Yet another embodiment of the invention encompasses crystalline DFX characterized by data selected from the group consisting of: a PXRD pattern having peaks at about 10.4, 11.9, and 15.6±0.2° degrees 2θ, a PXRD pattern depicted in FIG. 3, and combination thereof.

One embodiment of the invention encompasses a process for preparing crystalline DFX characterized by data selected from the group consisting of: a PXRD pattern having peaks at about 10.4, 11.9, and 15.6±0.2 degrees 2θ, and a PXRD pattern depicted in FIG. 3, and combination thereof comprising crystallizing DFX from a solvent mixture comprising acetone as the solvent, and water as the anti-solvent.

Another embodiment of the invention encompasses tetrahydrofuran (“THF”) solvate of DFX.

Yet another embodiment of the invention encompasses crystalline DFX characterized by data selected from the group consisting of: a PXRD pattern having peaks at about 6.8, 11.7, and 15.1±0.2 degrees 2θ, a PXRD pattern depicted in FIG. 4, and combination thereof.

One embodiment of the invention encompasses a process for preparing crystalline DFX characterized by data selected from the group consisting of: a PXRD pattern having peaks at about 6.8, 11.7, and 15.1±0.2 degrees 2θ, and a PXRD pattern depicted in FIG. 4, and combination thereof comprising providing a solution of DFX in THF, and removing the THF to obtain the said crystalline DFX.

One embodiment of the invention encompasses a pharmaceutical composition comprising a therapeutically effective amount of any one of the above crystalline DFX and combination thereof, and at least one pharmaceutically acceptable excipient.

Another embodiment of the invention encompasses a process for preparing pharmaceutical compositions of any one of the above crystalline DFX and combination thereof, comprising mixing a therapeutically effective amount of any one of the above crystalline forms of DFX and combination thereof with at least one pharmaceutically acceptable excipient.

Yet another embodiment of the present invention encompasses the use of any one of the above crystalline DFX, and combination thereof for the manufacture of a pharmaceutical composition.

### BRIEF DESCRIPTION OF THE FIGURES

FIG. 1 illustrates a PXRD pattern of crystalline form I of DFX

FIG. 2 illustrates a PXRD pattern of crystalline form II of DFX

FIG. 3 illustrates a PXRD pattern of crystalline form III of DFX

FIG. 4 illustrates a PXRD pattern of crystalline form IV of DFX

FIG. 5 illustrates a Microscopic picture of crystalline form IV of DFX

### DETAILED DESCRIPTION OF THE INVENTION

The present invention provides novel crystalline forms of DFX, processes for preparation thereof, and pharmaceutical compositions comprising the novel forms.

One embodiment of the invention encompasses crystalline DFX characterized by data selected from the group consisting of: a PXRD pattern having peaks at about 5.3, 10.6, and 13.9±0.2 degrees 2θ, a PXRD pattern depicted in FIG. 2, and combination thereof. This form can be designated form II.

The crystalline DFX form II can be further characterized by PXRD pattern having peaks at about 12.0, 15.6, 20.6, 21.2 and 23.0±0.2 degrees 2θ. In addition, the above crystalline can be further characterized by a weight loss of about 56.4 to about 69.8% as measured at temperatures of less or equal to about 123° C. by TGA. Preferably, the TGA measurement is done from a temperature of about 25° C. to about 123° C. The above crystalline can be characterized by any other method known to a skilled artisan, such as solid state NMR, and FTIR.

The above crystalline DFX form II has less than about 10% by weight, preferably, less than about 5% by weight, more preferably, less than about 1% by weight of crystalline DFX characterized by main PXRD peaks at 13.2, 14.1 and 16.6±0.2 degrees 2θ. designated form I. Preferably, the content of form I in form II is measured by PXRD, using any one of the peaks at 13.2 and 16.60±0.2 degrees 2θ.

The above crystalline DFX form II is prepared by a process comprising providing an aqueous solution of DFX having a basic pH, and reducing the pH to obtain an acidic pH to precipitate crystalline DFX form II.

Typically, the aqueous solution having a basic pH is provided by combining DFX and water to obtain a suspension, and admixing with an inorganic base to obtain the said solution.

Preferably, the starting DFX is obtained, for example, according to the process reported in IPCOM000146862D.

Typically, the inorganic base reacts with DFX transforming it to its salt, which is soluble in water. Preferably, the inorganic base is an alkali metal hydroxide, more preferably, NaOH, LiOH or KOH, most preferably, NaOH.

Typically, the reaction with an inorganic base provides a pH of at least about 8, preferably, of about 8 to about 14, more preferably, of about 10 to about 14, most preferably, of about 12 to about 14.

Typically, reducing the pH of the aqueous solution having a basic pH is done by admixing an acid with the said aqueous solution. Preferably, the acid is an inorganic acid, more preferably, HCl, nitric acid or sulfuric acid, most preferably, HCl.

Typically, a sufficient amount of acid is added so that an acidic pH is obtained. Typically, the reaction with the acid provides a pH of less than about 7. Preferably, the obtained acidic pH is of less than about 6, more preferably, of about 1 to about 6, most preferably, of about 5 to about 6. Typically, the salt of DFX reacts with the acid, providing again DFX, which precipitates in the form of crystals.

The process for preparing the above crystalline forms of DFX may further comprise recovering the crystalline DFX. The recovery can be done by a method that does not include drying, for example, by filtering the obtained crystals and washing.

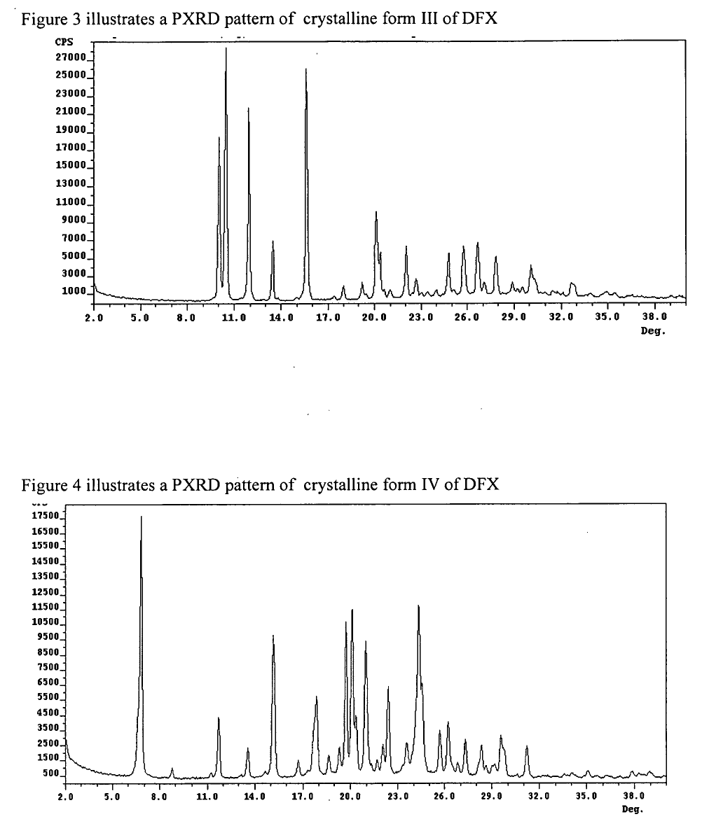

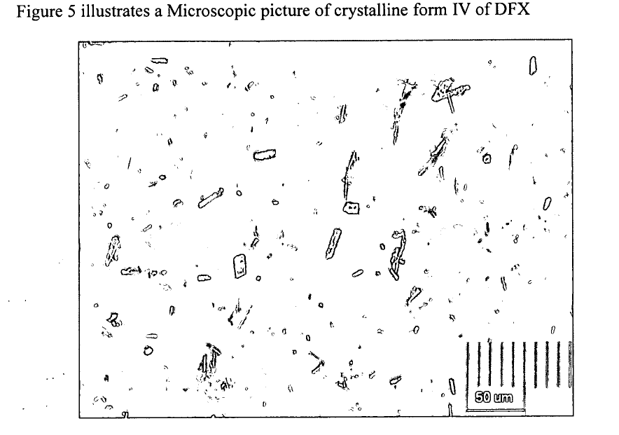

Another embodiment of the invention encompasses crystalline DFX characterized by data selected from the group consisting of: a PXRD pattern having peaks at about 10.4, 11.9, and 15.6±0.2 degrees 2θ, a PXRD pattern depicted in FIG. 3, and combination thereof. This form can be designated Form III.

The crystalline form III can be further characterized by PXRD pattern having peaks at about 10.0, 13.4, 21.9, 24.7, 25.7 and 27.8±0.2 degrees 2θ. In addition, the above crystalline form can be further characterized by a weight loss of about 21.7 to about 41.2% as measured at temperatures of less or equal to about 116° C. by TGA. Preferably, the TGA measurement is done from a temperature of about 25° C. to about 116° C. The above crystalline can be characterized by any other method known to a skilled artisan, such as solid state NMR, and FTIR.

The above crystalline DFX Form III has less than about 10% by weight, preferably, less than about 5% by weight, more preferably, less than about 1% by weight of form I of DFX. Preferably, the content of form I in form III is measured by PXRD, using any one of the peaks at about 13.2, 14.1 and 16.6±0.2 degrees 2θ.

The above crystalline form III is prepared by a process comprising crystallizing DFX from a mixture comprising acetone as the solvent, and water as the anti-solvent.

The crystallization is done by a process comprising dissolving DFX in acetone, and admixing the solution with water to obtain a suspension comprising the crystalline DFX.

Preferably, the dissolution is achieved at a temperature of about 15° C. to about 35° C., more preferably, at a temperature of about 20° C. to about 25° C.

Preferably, water is added to the solution, providing said suspension. Typically, the suspension is cooled to increase the yield of said crystalline DFX. Preferably, the suspension is cooled to a temperature of about 8° C. to about 2° C. Preferably, cooling is conducted for a period of 2 to about 48 hours, more preferably, for about 3 to about 10 hours.

The process for preparing the above crystalline may further comprise, recovering the crystalline DFX. The recovery can be done by a method that does not include drying, for example, by filtering the obtained crystals.

Yet another embodiment of the invention encompasses a tetrahydrofuran solvate of DFX.

Yet another embodiment of the invention encompasses crystalline DFX characterized by data selected from the group consisting of: a PXRD pattern having peaks at about 6.8, 11.7, and 15.1±0.2 degrees 2θ, a PXRD pattern as depicted in FIG. 4, and combination thereof. This form can be designated Form IV.

The crystalline form can be further characterized by PXRD pattern having peaks at about 13.5, 17.8, 19.7, 20.1, 21.0, 22.4 and 24.3±0.2 degrees 2θ. In addition, the above crystalline can be further characterized by a weight loss of about 15.4 to about 16.7% as measured at temperatures of about less or equal to 175° C. by TGA. Preferably, the TGA measurement is done from a temperature of about 25° C. to about 175° C. The said crystalline form is a solvated form of DFX, preferably, a tetrahydrofuran solvate of DFX. The above solvate is a preferred intermediate for purifying DFX; since its crystals are characterized by a small surface area and thus absorb fewer impurities from the solution. The above crystalline form can be characterized by any other method known to a skilled artisan, such as solid state NMR, and FTIR.

The above crystalline DFX has less than about 10% by weight, preferably, less than about 5% by weight, more preferably, less than about 1% by weight of form I of DFX. Preferably, the content of form I in form IV is measured by PXRD, using any one of the peaks at about 10.0 and 14.1°±0.2 degrees 2θ.

The above crystalline form IV is prepared by a process comprising providing a solution of DFX in THF, and removing the THF to obtain the said crystalline DFX.

Preferably, dissolution is achieved at a temperature of about 15° C. to about 35° C., more preferably, at a temperature of about 20° C. to about 25° C.

Preferably, removing the solvent is done by evaporation at a temperature of about 30° C. to about 50° C., more preferably, at about 50° C. Preferably, evaporation is done under reduced pressure, providing an oil which solidifies in the form of crystals. Preferably, the reduced pressure is of about 20 to about 250 mbar, more preferably, of about 100 to about 140 mbar.

The present invention also provides transformation processes; wherein one form of crystalline DFX transforms into another or into a mixture of DFX crystalline forms by drying. The crystalline form of DFX of the present invention can be transformed into crystalline Form I or into its mixtures with other crystalline forms.

Preferably, the drying is performed at about room temperature to a temperature of about 125° C., preferably, at a temperature of about 115° C. to about 125° C. As used herein, the term “room temperature” refers to a temperature of about 15° C. to about 35° C., preferably, to about 20° C. to about 25° C. Preferably, drying is done for about 25 minutes to about over night, preferably for about 25 to about 35 minutes. Typically, drying time is depended on the drying temperature, for example, drying at a room temperature is done for overnight, preferably, for about 10 hours to about 20 hours. As used herein, the term “over night” refers to a period of about 10 hours to about 20 hours, preferably, of about 14 hours to about 16 hours.

In a preferred embodiment drying crystalline DFX form II at a temperature of about room temperature to about 120° C., provides a mixture of Form II and Form I.

In another preferred embodiment drying crystalline DFX form III at a temperature of about room temperature to about 120° C., provides form I.

In another preferred embodiment drying crystalline DFX form IV at a temperature of about room temperature to about 120° C., provides form I, preferably, drying is done at a temperature of about 115° C. to about 120° C.

One embodiment of the invention encompasses a pharmaceutical composition comprising a therapeutically effective amount of any one of the above crystalline forms of DFX and combination thereof, and at least one pharmaceutically acceptable excipient.

Another embodiment of the invention encompasses a process for preparing pharmaceutical compositions of any one of the above crystalline forms of DFX and combination thereof, comprising mixing a therapeutically effective amount of any one of the above crystalline forms of DFX and combination thereof, with at least one pharmaceutically acceptable excipient.

Yet another embodiment of the present invention encompasses the use of any one of the above crystalline forms of DFX and combination thereof, for the manufacture of a pharmaceutical composition.

### EXAMPLES

### PXRD Method

ARL X-ray powder diffractometer model X'TRA-030, equipped with Cu irradiation source (λ=1,54178 \acute{Å} (Angström)), Peltier detector, round standard aluminium sample holder with round zero background quartz plate was used. Scanning parameters: Range: 2-40 deg. 2 θ, continuous scan, Rate: 3 deg./min. The accuracy of peak positions is defined as +/−0.2 degrees due to experimental differences in instrumentation, sample preparation, etc.

### Thermal Gravimetric Analysis (TGA)

TGA/SDTA 851^{e}, Mettler Toledo, sample weight 7-20 mg. Heating rate: 10° C./min., N₂ stream flow rate: 50 ml/min., Scan range: 30-250° C.

### Example 1

### Preparation of Crystalline DFX Form II

Deferasirox (0.5 g) was suspended in water (30 ml) at room temperature. Solid NaOH was added to the suspension under stirring until obtaining a clear solution having a pH greater than 10. The pH of the solution was adjusted to about 6 with diluted aqueous HCl. The precipitated DFX was filtered off after 30 minutes stirring, and washed with water. Polymorphic form of wet sample was determined by the X-Ray Powder Diffraction and found to be crystalline DFX form II.

### Example 2

### Preparation of a Mixture of Form I and Crystalline DFX Form II

The crystalline DFX form II was left in the air at room temperature for overnight to allow drying.

### Example 3

### Preparation of a Mixture of Form I and Crystalline DFX Form II

DFX form II was heated at 120° C. for 30 minutes.

### Example 4

### Preparation of Crystalline DFX Form III

Deferasirox (0.5 g) was dissolved in acetone (35 ml) at room temperature. Water (35 ml) was added in one portion. The Suspension was cooled in a fridge, for overnight. The precipitated DFX was filtered off without washing. Polymorphic form of wet sample was determined by the X-Ray Powder Diffraction.

### Example 5

### Preparation of Crystalline DFX Form I

DFX form III was left in the air at room temperature overnight to allow drying.

### Example 6

### Preparation of Crystalline DFX Form I

DFX form III was heated at 120° C. for 30 minutes.

### Example 7

### Preparation of Crystalline DFX Form IV

Deferasirox (0.5 g) was dissolved in THF (10 ml) at room temperature. The solution was evaporated at a temperature of about 30° C. to about 50° C. under reduced pressure at about 20 mbar to about 250 mbar to produce an oily residue. The residue solidified after some minutes. The polymorphic form of the sample was determined by X-Ray Powder Diffraction, which showed that the sample was crystallized in a new form.

### Example 8

### Preparation of Crystalline Form I of DFX

DFX form IV was heated to 120° C. for 30 minutes, providing form I.

### Example 9

### Preparation of form I of DFX (IPCOM000146862D)

2-(2-hydroxyphenyl)benz(e)[1,3]oxazin-4-one (15.0 g) and 4-hydrazino-benzoic acid (10.5 g) are boiled under reflux in ethanol (225 ml). The reaction is checked for completion after 2 hours by Thin Layer Chromatography (TLC). If the reaction is not complete, the reaction mixture is stirred for an additional hour and the conversion is checked again until it is complete. If the reaction is complete, the mixture is cooled to room temperature and the precipitated solid material is filtered off, washed with ethanol and dried in vacuum. Yield: 82.5%.

What is claimed is:

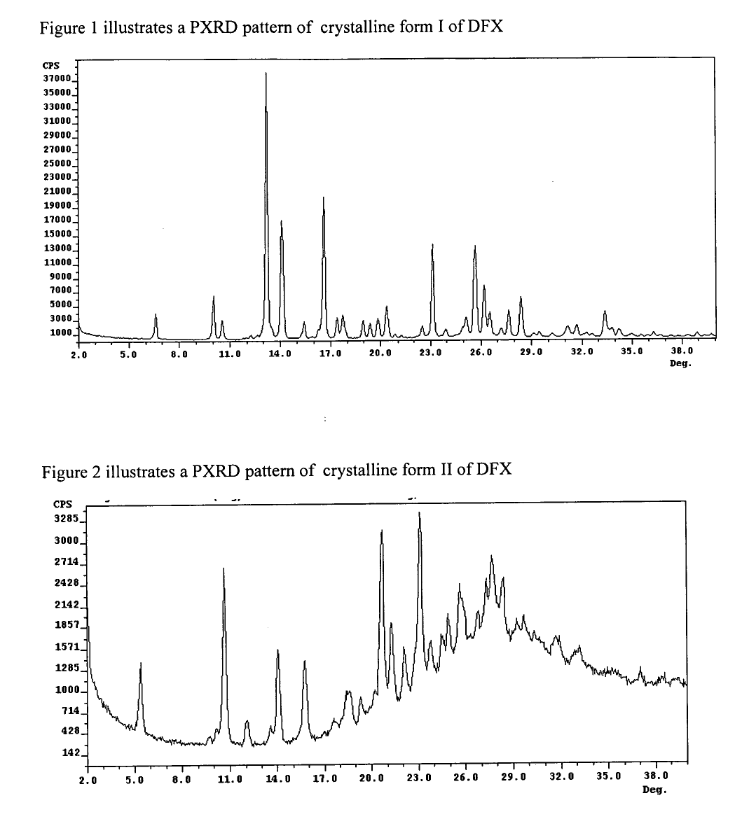

1. Crystalline deferasirox characterized by data selected from the group consisting of: a PXRD pattern having peaks at about 5.3, 10.6, and 13.9±0.2 degrees 2θ, a PXRD pattern depicted in FIG. 1, and combination thereof.

2. The crystalline deferasirox according to claim 1 characterized by a PXRD pattern having peaks at about 5.3, 10.6, and 13.9±0.2 degrees 2θ.

3. The crystalline deferasirox according to claim 2, further characterized by a PXRD pattern having peaks at about 12.0, 15.6, 20.6, 21.2 and 23.0±0.2 degrees 2θ.

4. The crystalline deferasirox according to claim 1, characterized by a PXRD pattern depicted in FIG. 1.

5. The crystalline deferasirox according to claim 1, further characterized by a weight loss of about 56.4 to about 69.8% as measured at temperatures of less or equal to about 123° C. by TGA.

6. Crystalline deferasirox according to claim 1 containing less than about 10% by weight of crystalline deferasirox characterized by main PXRD peaks at 13.2, 14.1 and 16.6±0.2.

7. A process for preparing the crystalline deferasirox of claim 1, comprising the steps of (a) providing a solution of DFX in water having a basic pH, and (b) reducing the pH to obtain an acidic pH to precipitate the crystalline DFX.

8. The process according to claim 7, wherein the solution of DFX in water having a basic pH is provided by combining DFX to obtain a suspension and admixing with an organic base.

9. The process according to claim 8, wherein the inorganic base is an alkali metal hydroxide.

10. The process according to claim 9, wherein the alkali metal hydroxide is NaOH, LiOH or KOH.

11. The process according to claim 7, wherein the pH of the aqueous solution is of least about 8.

12. The process according to claim 7, wherein the pH is reduced by admixing the aqueous solution having basic pH with an acid.

13. The process according to claim 12, wherein the acid is an inorganic acid.

14. The process according to claim 13, wherein the inorganic acid is HCl, nitric acid or sulfuric acid.

15. The process according to claim 7, wherein the acidic pH is of less than about 7.

16. The process according to claim 7, further comprising recovering the said crystalline DFX.

17. Crystalline DFX characterized by data selected from the group consisting of: a PXRD pattern having peaks at about 10.4, 11.9, and 15.6±0.2° degrees 2θ, a PXRD pattern depicted in FIG. 2, and combination thereof.

18. The crystalline deferasirox according to claim 17, characterized by a PXRD pattern having peaks at about 10.4, 11.9, and 15.6±0.2 degrees 2θ.

19. The crystalline deferasirox according to claim 18, further characterized by a PXRD pattern having peaks at about 10.0, 13.4, 21.9, 24.7, 25.7 and 27.8±0.2 degrees 2θ.

20. The crystalline deferasirox according to claim 17, characterized by a PXRD pattern as depicted in FIG. 2.

21. The crystalline deferasirox according to claim 17, further characterized by a weight loss of about 21.7% to about 41.2% as measured at temperatures of less or equal to about 116° C. by TGA.

22. The crystalline deferasirox according to claim 17, containing less than about 10% by weight of crystalline deferasirox characterized by main PXRD peaks at 13.2, 14.1 and 16.6±0.2.

23. A process for preparing the crystalline deferasirox of claim 17, comprising crystallizing DFX from a solvent mixture comprising acetone as a solvent and water as an anti solvent.

24. The process for preparing the crystalline deferasirox of claim 23, comprising the steps of (a) providing a solution of deferasirox in acetone, and (b) admixing said solution with water to obtain a suspension comprising deferasirox.

25. The process according to claim 24, wherein the dissolution of deferasirox in acetone is achieved at a temperature of about 15° C. and about 35° C.

26. The process according to claim 24, wherein, water is added to the solution providing the said suspension.

27. The process according to claim 23, further comprising recovering the said crystalline DFX.

28. Crystalline THF solvate of DFX.

29. Crystalline DFX characterized by data selected from the group consisting of: a PXRD pattern having peaks at about 6.8, 11.7, and 15.1±0.2 degrees 2θ, a PXRD pattern depicted in FIG. 3, and combination thereof.

30. The crystalline deferasirox according to claim 29, characterized by a PXRD pattern having peaks at about 6.8, 11.7, and 15.1±0.2 degrees 2θ.

31. The crystalline deferasirox according to claim 30, further characterized by a PXRD pattern having peaks at about 13.5, 17.8, 19.7, 20.1, 21.0, 22.4 and 24.3±0.2 degrees 2θ.

32. The crystalline deferasirox according to claim 29, characterized by a PXRD pattern depicted in FIG. 3.

33. The crystalline deferasirox according to claim 29, further characterized by a weight loss of about 15.4% to about 16.7% as measured at temperatures of less or equal to about 175° C. by TGA.

34. Crystalline deferasirox according to claim 29, containing less than about 10% by weight of crystalline deferasirox characterized by main PXRD peaks at 13.2, 14.1 and 16.6±0.2.

35. The crystalline deferasirox according to claim 29, wherein the crystalline form is a tetrahydrofuran solvate.

36. A process for preparing the crystalline deferasirox of claim 29, comprising providing a solution of DFX in tetrahydrofuran, and removing the tetrahydrofuran to obtain the said crystalline DFX.

37. A process for preparing the crystalline deferasirox of claim 36, comprising the steps of (a) providing a solution of deferasirox in tetrahydrofuran, (b) removing the tetrahydrofuran by evaporation, thereby producing a residual oil, and (c) allowing said oil to solidify.

38. The process according to claim 37, wherein dissolution of deferasirox is achieved at a temperature of about 15° C. to about 35° C.

39. The process of claim 37, wherein the tetrahydrofuran is removed by evaporation under a pressure of about 100 mbar to about 140 mbar.

40. The process according to claim 36, further comprising recovering the said crystalline DFX.

41. A process for producing crystalline deferasirox characterized by a PXRD pattern with peaks at about 13.2, 14.1 and 16.6±0.2 degrees 2θ, comprising drying crystalline deferasirox selected from the group consisting of: crystalline deferasirox of claim 1, crystalline deferasirox of claim 17, and crystalline deferasirox of claim 29 at a temperature from about room temperature to about 125° C.

42. The process according to claim 41, wherein crystalline deferasirox of claim 29 is dried at a temperature of about 115° C. to about 120° C.

43. A pharmaceutical composition comprising a therapeutically effective amount of a crystalline deferasirox according to any one of claims 1, 17, or 29, and mixtures thereof and at least one pharmaceutically acceptable excipient.

44. The use of a crystalline deferasirox according to any one of claims 1, 17, or 29, and mixtures thereof in the manufacture of a pharmaceutical composition for the treatment of iron overload.

45. A process for preparing pharmaceutical compositions of any one of the crystalline forms of DFX of claims 1, 17, or 29 and mixtures thereof, comprising mixing a therapeutically effective amount of any one of the crystalline forms of DFX of claims 1, 17, or 29 and mixtures thereof, with at least one pharmaceutically acceptable excipient.

46. Use of a crystalline form of DFX according to any of claims 1 to 6, 17 to 22, and 28 to 35 as an intermediate for the preparation of crystalline DFX Form I.
